(19) United States
(12) Patent Application Publication
(10) Pub. No.: US 2018/0250160 A1
(43) Pub. Date: Sep. 6, 2018

(54) Knee and Foot Therapy Apparatus

(71) Applicant: Gary Lawrence Johnston, Cowarts, AL (US)

(72) Inventor: Gary Lawrence Johnston, Cowarts, AL (US)

(21) Appl. No.: 15/450,045

(22) Filed: Mar. 6, 2017

(51) Int. Cl. A61F 7/02; A61F 7/00

(52) U.S. Cl. CPC A61F 7/02; A61F 2007/0045; A61F 2007/0042; A61F 7/007

(57) ABSTRACT

A knee and foot therapy apparatus is provided which enables heat therapy to be easily applied to the knee and foot parts of the body. The apparatus comprises a knee support member, a foot support member, a support connection member, and a heat transfer member. The support connection member is for adjustably connecting the knee support member and the foot support member together to allow the apparatus to be easily adjusted for different user leg lengths. The user will place the back of their knee or knees upon the top of the knee support member, and place the heel of their foot or feet upon the top of the foot support member. The heat transfer member has a knee cover member and a foot cover member mounted to the knee support member and to the foot support member respectively. These cover members are heated by an electrical source, and thus provide heat therapy to both the knee and foot parts of the body. Other configurations of the device may include multiple knee and foot covers, a separate and independently operating knee and foot heat transfer member, and a collapsing feature which allows the apparatus to be more easily stored and transported

### BACKGROUND OF THE INVENTION

This invention relates to a knee and foot therapy apparatus which has a knee support member and a foot support member connected to one another A knee heating member and a foot heating member are mounted upon the support members to allow the user to perform therapy routines on the knee and foot portions of the body while in a relatively relaxed position. The ability to heat the knee and foot portions of the body allows for an easy and comfortable form of heat therapy for these areas which are sore or have been traumatized due to an accident or illness.

### SUMMARY AND OBJECTS OF THE INVENTION

It is the object of this invention to provide a therapeutic apparatus which may provide the user an efficient and inexpensive means for heating the knee and foot portions of the body. The main purpose of this application is to demonstrate an apparatus which performs the stated function, and to demonstrate the many options and configurations this apparatus may take on.

Briefly stated, the apparatus that forms the basis of the present invention comprises a knee support member, a foot support member, a support connection member, and at least one heat transfer member. The user will place the back of their knee or knees upon the top of the knee support member, and place the heel of their foot or feet upon the top of the foot support member. The support connection member is for adjustably connecting the knee support member and the foot support member together. It also allows the apparatus to be easily adjusted for different user leg lengths. The heat transfer member is used to provide heat to both the knee and foot parts of the body, either individually or simultaneously. The heat transfer member includes knee cover members that may wrap around the knee or knees, and foot cover members that may cover most of the foot of the user. Heat from an electrical source is used to provide heat to the cover members, and thus provide heat to the knee and foot areas. The heat or warmth applied to the knee and foot provides a therapeutic relief to the related joints which are sore or which have been traumatized due to an accident or illness.

One optional feature of the device is that the heat transfer member may include two or more separate heat sources, which would allow different amounts of heat to be applied to the knee and feet, to the individual knees, or to the individual feet. Another optional feature is to have the apparatus collapseable by having the support connection member collapseable. Also, portions of the heat transfer member being placed within a hollow portion of one or both of the knee support member and foot support member, so that they apparatus may be stored more easily and transported more easily.

### BRIEF DESCRIPTION OF THE DRAWINGS

FIG. 1A is a front view of the knee and foot therapy apparatus.

FIG. 1B is a top view of the knee and foot therapy apparatus.

FIGS. 1C and 1D are side views of the knee and foot therapy apparatus, showing the knee heating portion of the apparatus.

FIG. 1E is a side view of the knee and foot therapy apparatus, showing the foot heating portion of the apparatus.

FIG. 2A is a front view of the knee support member, foot support member, and support connection member of the knee and foot therapy apparatus.

FIG. 2B is a top view of the knee support member, foot support member, and support connection member of the knee and foot therapy apparatus.

FIGS. 2C and 2D are side views of the knee support member, foot support member, and support connection member of the knee and foot therapy apparatus.

FIG. 3A is a front view of the knee heat transfer member of the knee and foot therapy apparatus.

FIG. 3B is a top view of the knee heat transfer member of the knee and foot therapy apparatus.

FIG. 3C is a side view of the knee heat transfer member of the knee and foot therapy apparatus.

FIG. 4A is a front view of the foot heat transfer member of the knee and foot therapy apparatus.

FIG. 4B is a top view of the foot heat transfer member of the knee and foot therapy apparatus.

FIG. 4C is a side view of the foot heat transfer member of the knee and foot therapy apparatus.

FIG. 5 is a side view demonstrating the basic operation of the knee and foot therapy apparatus.

FIG. 6A is a front view demonstrating one of the collapsing features of the knee and foot therapy apparatus.

FIGS. 6B and 6C are side views demonstrating another of the collapsing features of the knee and foot therapy apparatus.

### DETAILED DESCRIPTION OF THE PREFERRED EMBODIMENT

Before explaining in detail the present invention, it is to be understood that the invention is not limited in its application to the details of construction or arrangement of parts illustrated in the accompanying drawings, since the invention is capable of other embodiments and of being practiced or carried out in various ways. Also, it is to be understood that the phraseology and terminology employed herein is for the purpose of description, and not limitation.

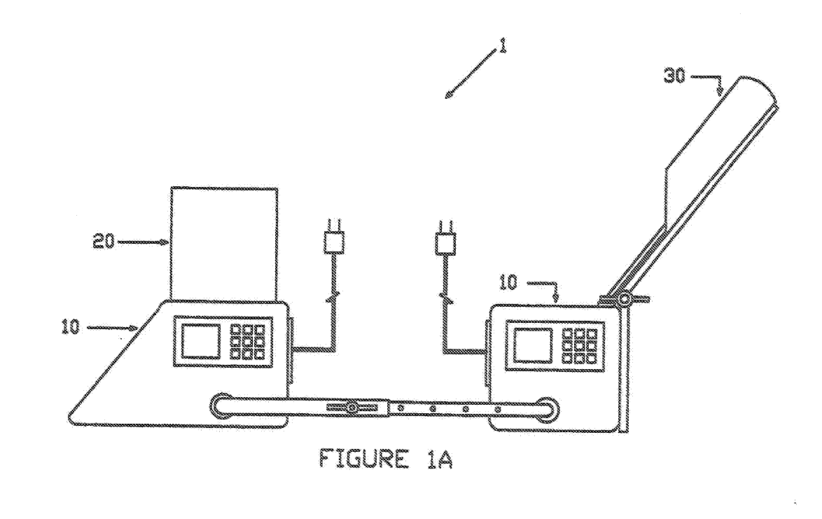

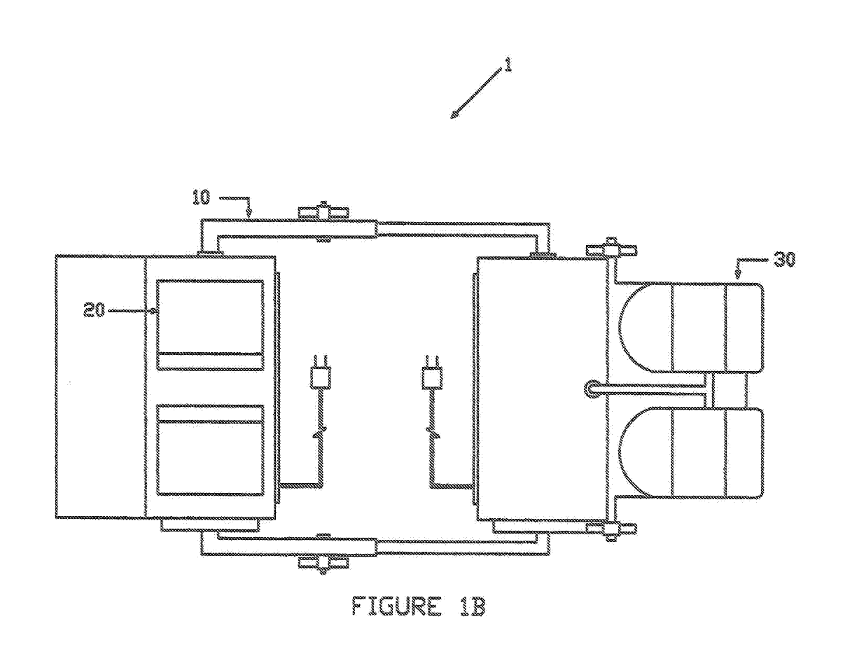

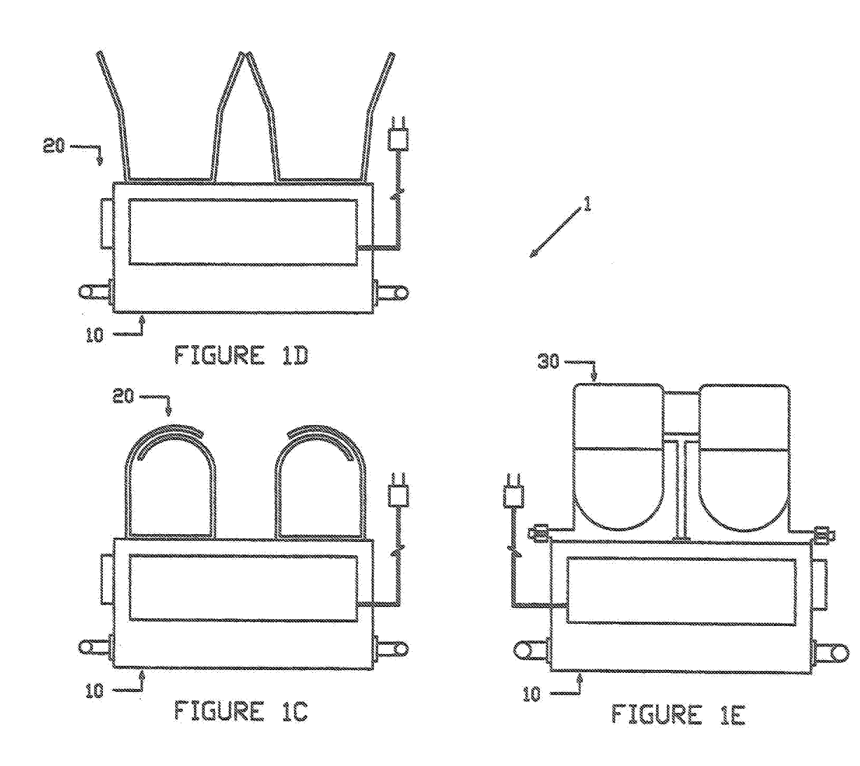

As best can be seen by references to the drawings, and in particular to FIGS. 1A-1C, the knee and foot therapy apparatus that forms the basis of the present invention is designated generally by the reference numeral 1, and includes a knee and foot support structure 10, a knee beat transfer member 20, and a foot heat transfer member 30. The heat transfer members 20 and 30 mount upon the knee and foot support structure 10. The knee heat transfer member 20 and the foot transfer member 30 are shown as separate components, but a single heat transfer system which heats both the knee and foot could be utilized.

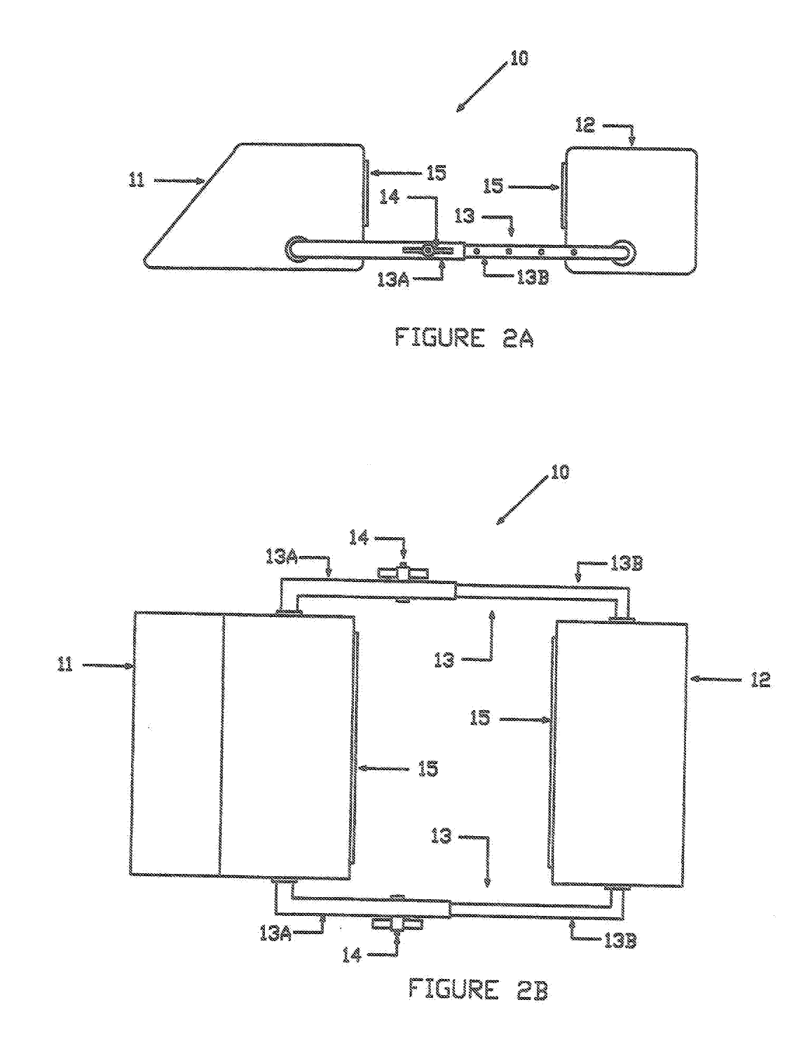

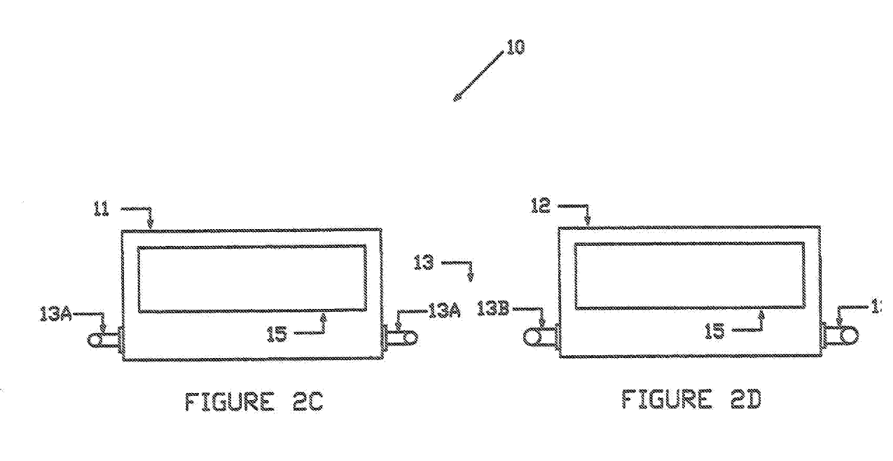

As may be seen in FIGS. 2A-2D, the knee and foot support structure 10 may have a generally elongated-shaped knee support member 11 and a generally elongated-shaped foot support member 12. Each has an upper portion, a bottom portion, and sides. The knee support member 11 may have sufficient length so that the user may rest the back of their knee or knees on the upper portion of the knee support member 11, with the bottom portion being supported by an underneath surface, such as a couch, bed, floor, etc. The foot support member 12 may have sufficient length so that the user may also rest the heel of their foot or feet on the upper portion of the foot support member 20, with the bottom portion also being supported by an underneath surface, such as a couch, bed, floor, etc. The knee support member 11 and foot support member 12 may have a hollow inside with a flap member 15, which may be pulled open so that items may be stored within.

A support connection member 13 may also be included which mounts at one end to knee support member 11 and at its other end to foot support member 12. It connects the knee support member 11 and foot support member 12 together. The support connection member 13 may be comprised of two separate components, knee support connection member 13A and foot support connection member 13B. The knee support connection member 13A mounts to knee support member 11, while foot support connection member 13B mounts to foot support member 12. The support connection members 13A and 13B connect to one another in a telescoping-like fashion, such that the relative distance between the knee support member 11 and the foot support member 12 may be selectively varied. They may be secured together using a commonly used wing nut and bolt assembly 14. Preferably, each side of the knee support member 11 is connected to the respective side of foot support member 12 by a support connection member 13, as shown in the figures. However, it is possible to connect only one of the respective sides together.

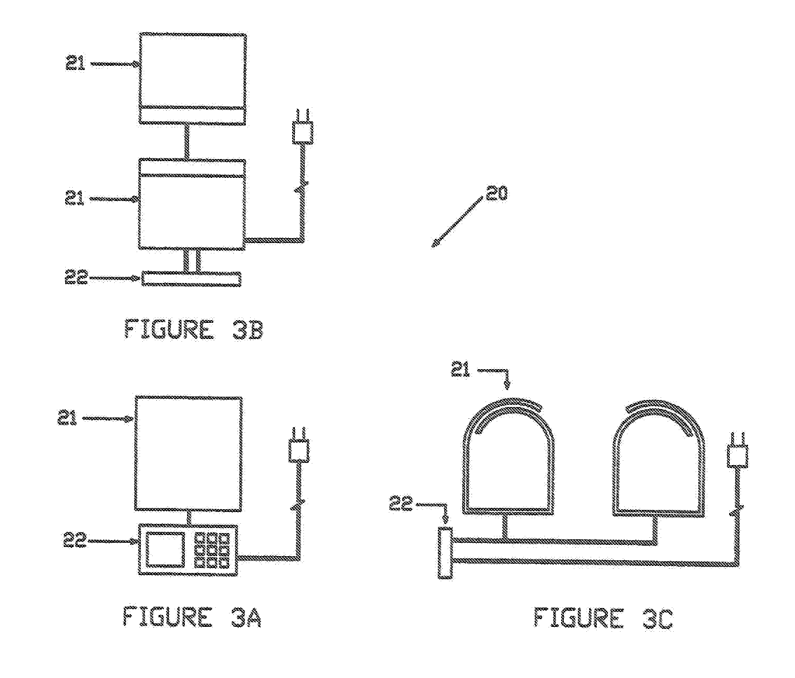

As may be seen in FIGS. 3A-3C, the knee heat transfer member 20 is used to supply heat to the knee areas of the body. The knee heat transfer member 20 includes at least one knee cover member 21 which attaches to the general top surface of knee support member 11, and may be placed around the knee to transfer heat to the back, side, and top areas of the knee. Knee cover member 21 may be a type of wrapping object with ends which attach together using a common attachment means, such as velcro.

Knee heat transfer member 20 also has an electrical heating assembly 22, which is a commonly found type of device found in heat pads, electric blankets, etc. The assembly has a conventional heat controller unit which plugs into an electrical outlet. Coming from the heat controller may be heating elements which extend within the fabric of the knee cover member 21, and are used to provide heat to the cover member. The heat elements may be arranged within the knee cover member 21 such that heat is supplied to the top, side, and bottom portions of the knee. The conventional heat controller unit of the electrical heating assembly may attach in some manner to the knee support member 11, or may be a separate item as is commonly found in electrical blankets and heat pads. It is used to turn on, turn off, and adjust the heat generated by the electrical heating assembly 22.

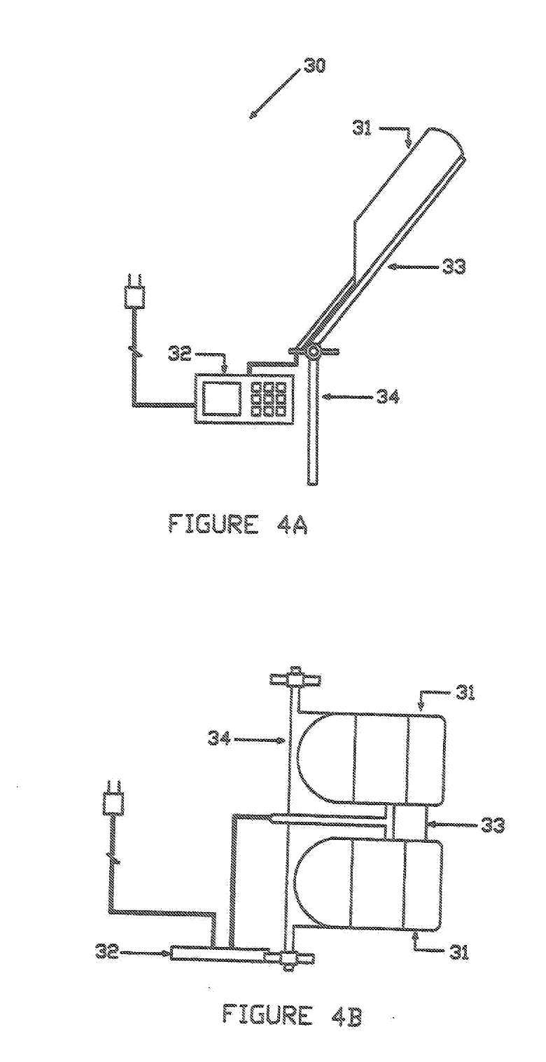

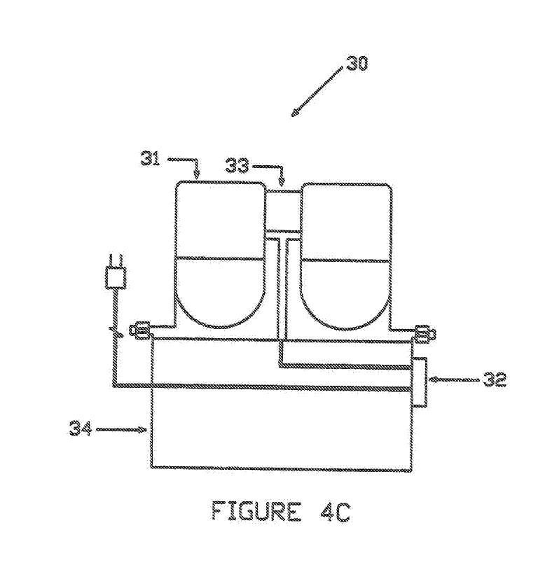

The foot heat transfer member 30 is shown in FIGS. 4A-4C, and has at least one foot cover member 31 which attaches to the foot support member 12 through top support structure member 33 and bottom support structure member 34. As may be seen, bottom support structure member 34 is rigidly attached to the back side of foot support member 12. Top support structure member 33 is pivotally mounted to bottom support structure member 34 such that the top support structure member 33 may be secured at different angles by a means, such as a wing nut and bolt assembly. Foot cover member 31 is mounted to the front part of top support structure member 33, and may be a pouch-like object into which the user easily slides their foot, with the pouch covering a large portion of the foot, including the toes. Or, the foot cover member 31 may be similar to the knee cover 21 and wrap around a portion of the foot.

Foot heat transfer member 30 also has an electrical heating assembly 32, which is a commonly found type of device found in heat pads, electric blankets, etc. Again, the assembly may have a conventional heat controller which plugs into an electrical outlet. Coming from the heat controller may be heating elements which extend within the fabric of the foot cover member 31, and are used to provide heat to the cover member. The heat elements may be arranged within the foot cover member 31 such that heat is supplied to the top, side, and bottom portions of the foot. The conventional heat controller of the electrical heating assembly may attach in some manner to the foot support member 12, or may be a separate item as is commonly found in electrical blankets and heat pads. It is used to turn on, turn off, and adjust the heat generated by the electrical heating assembly 22.

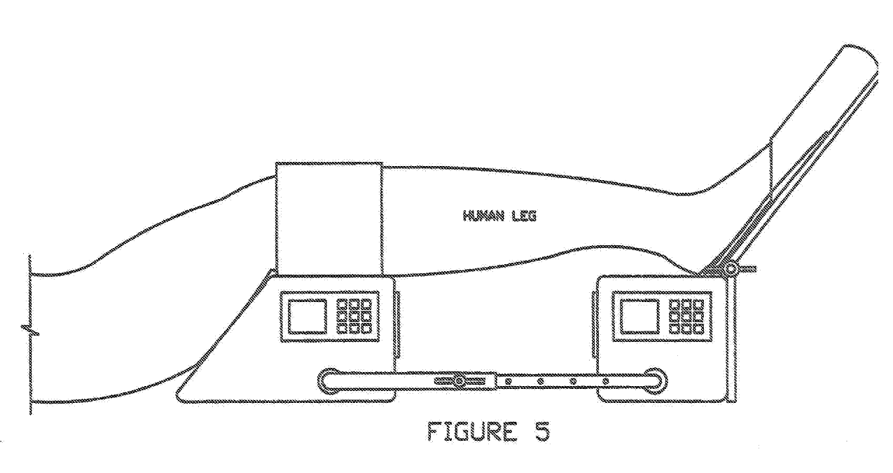

FIG. 5 demonstrates the operation of the knee and foot therapy apparatus. As shown the user may position themself in a sitting or lying position upon a relatively flat surface, such as a bed or couch. The apparatus would be placed in a relatively horizontal position upon the same flat surface. The user may then place their knee or knees upon the knee support member, and wrap the knee cover member(s) around the knee or knees. The user may also slide their foot or feet into the foot cover member(s), with the heel of the foot or feet resting upon the foot support member. The user may adjust the amount of heat to be applied to the knee or foot using the respective heat transfer member. Having two heat transfer members increases the flexibility of the apparatus, but it is possible to utilize only one to heat both the knee and foot. Also, it is preferable to have two separate knee cover members and two separate foot cover members as shown, so that the user can apply heat to both at once. However, it is also possible to have an apparatus which utilizes only one of each.

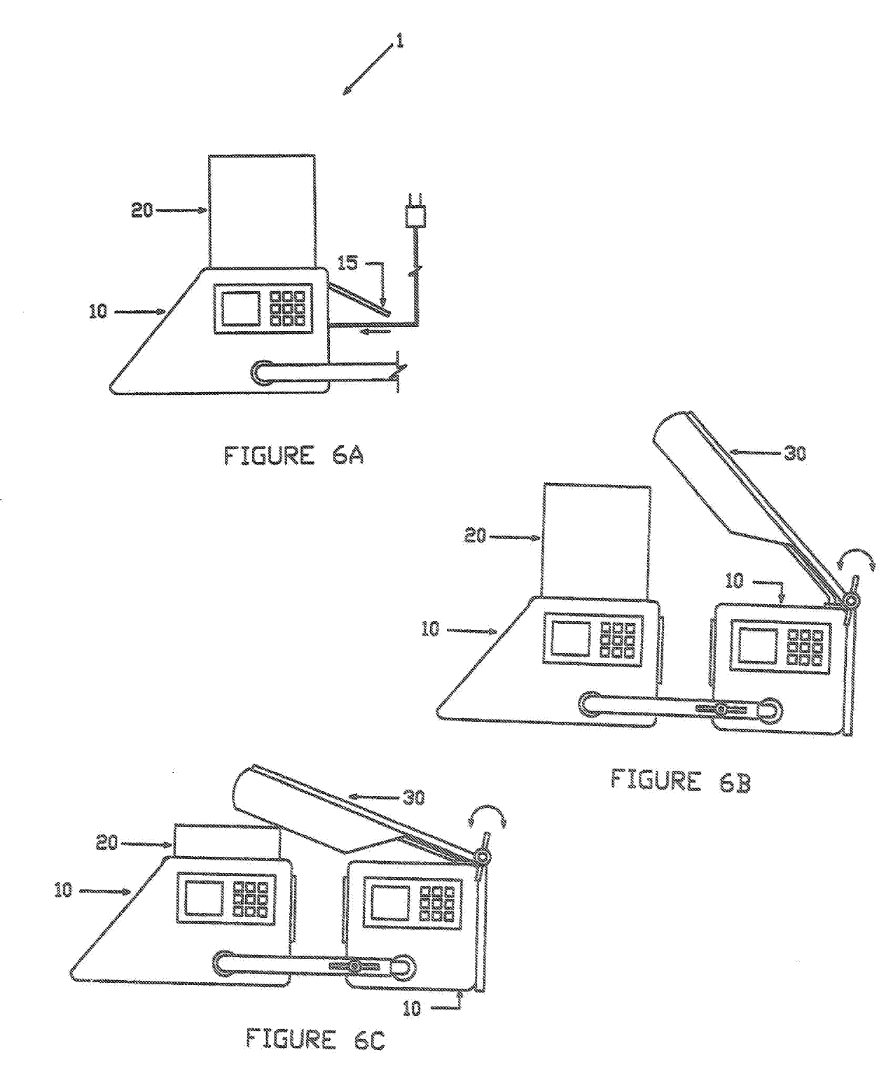

FIGS. 6A-6C demonstrates the collapsing feature which may be a part of the knee and foot therapy apparatus. As shown in FIG. 6A and previously mentioned, both the knee support member and foot support member of the knee and foot support structure 10 may have a hollow inside with a flap member which may be opened so that items, such as the electrical cord of the electrical heating assembly, may be stored within. Also, if the heat controller part of the electrical heating assembly is not mounted to the respective support members, then it also may be stored in the hollow inside. FIGS. 6B and 6C demonstrate the adjustable feature of the support connection member of knee and foot support structure 10. This feature would allow the relative distance between the knee support member and the foot support member to be selectively varied. This makes the device flexible so that different people having different leg lengths may utilize the apparatus. The apparatus could then be used by adults and by children alike. This feature to decrease the overall length of the apparatus should also help with storage and transport. The figures also show how the knee cover member may be made flatter, and the foot cover member folded downward, to reduce the overall height of the apparatus. This would also be useful for storage and transport purposes.

Many variations of the knee and foot therapy apparatus exist, along with the configurations described above. While it will be apparent that the preferred embodiment of the invention herein disclosed is well calculated to fulfill the objects above stated, it will be appreciated that the invention is susceptible to modification, variation, and change without departing from the proper scope or fair meaning of the subjoined claims.

I claim:

1. A knee and foot therapy apparatus comprising:
a knee support member having an upper portion and a bottom portion;
a foot support member having an upper portion and a bottom portion;
a support connection member for connecting said knee support member and said foot support member together;
a heat transfer member comprising at least one knee cover member mounted to the general upper portion of said knee support member, and at least one foot cover member mounted to the general upper portion of said foot support member, and at least one electrical heating assembly operative connected to said knee cover member and said foot cover member for transferring heat to said knee cover member and foot cover member;
whereby a user may position themself with at least one leg extending in a generally horizontal position, with the bottom portions of said knee support member and said foot support member being placed upon a generally horizontal surface structure, such that the user may place the back of the knee or knees upon the upper portion of said knee support member and cover at least a part of their knee or knees with said knee cover member, and place the heel part of the foot or feet upon the upper portion of said foot support member and cover at least apart of the foot or feet with said foot cover member, and use said electrical heating assembly to provide heat to the knee or knees and foot or feet of the user for therapeutic purposes.

2. The knee and foot therapy apparatus as claimed in claim 1, said knee support member having a generally elongated shape such that the underside of both knees may be supported.

3. The knee and foot therapy apparatus as claimed in claim 1, said foot support member having a generally elongated shape such that the heel area of both feet may be supported.

4. The knee and foot therapy apparatus as claimed in claim 1, said support connection member being adjustable such that the relative distance between said knee support member and said foot support member may be varied.

5. The knee and foot therapy apparatus as claimed in claim 1, said knee cover member and said foot cover member being cloth-like components, said electrical heating assembly having electrical heating elements located inside of said cloth-like knee cover member and said cloth-like foot cover member, said electrical heating assembly also having at least one heat adjustment component for connecting said electrical heating elements to an electrical current source, said electrical current source used to supply heat to the knee and foot cover members, said heat adjustment component also used to vary the amount of electrical current utilized so that the amount of heat supplied to the knee and foot cover members may be varied.

6. The knee and foot therapy apparatus as claimed in claim 2, said knee support member being hollow inside and capable of storing at least part of said electrical heating assembly.

7. The knee and foot therapy apparatus as claimed in claim 3, said foot support member being hollow inside and capable of storing at least part of said electrical heating assembly.

8. The knee and foot therapy apparatus as claimed in claim 1, said foot cover member being pivotally mounted to said foot support member such that said foot cover member may be positioned and secured at different angles.

9. The knee and foot therapy apparatus as claimed in claim 4, said support connection member being substantially adjustable such that said knee support member and said foot support member may be secured in close proximity to one another for storage and transport purpose.

10. A knee and foot therapy apparatus comprising:
a knee support member having an upper portion and a bottom portion;
a foot support member having an upper portion and a bottom portion;
a support connection member for connecting said knee support member and said foot support member together;
a knee heat transfer member comprising a knee cover member mounted to the general upper portion of said knee support member, and an electrical heating assembly operative connected to said knee cover member for transferring heat to said knee cover member;
a foot heat transfer member comprising a foot cover member mounted to the general upper portion of said foot support member, and an electrical heating assembly operative connected to said foot cover member for transferring heat to said foot cover member;
whereby a user may position themself with atheir legs extending in a generally horizontal position, with the bottom portions of said knee support member and foot support member being placed upon a generally horizontal surface structure, such that the user may place the back of the knees upon the upper portion of said knee support member and cover at least a part of their knees with said knee cover member, and place the heel portion of the feet upon the upper portion of said foot support member and cover at least a part of the feet with said foot cover member, and use said electrical heating assembly to provide heat to the knee and feet of the user for therapeutic purposes.

11. The knee and foot therapy apparatus as claimed in claim 10, said knee support member having a generally elongated shape such that the underside of both knees may be supported.

12. The knee and foot therapy apparatus as claimed in claim 10, said foot support member having a generally elongated shape such that the heel area of both feet may be supported.

13. The knee and foot therapy apparatus as claimed in claim 10, said support connection member being adjustable such that the relative distance between said knee support member and said foot support member may be varied.

14. The knee and foot therapy apparatus as claimed in claim 10, said knee cover member and said foot cover member being cloth-like components, each of said electrical heating assemblies having electrical heating elements located inside of respective said cloth-like knee cover member and said cloth-like foot cover member, each of said respective electrical heating assemblies also having an adjustment component for connecting said electrical heating elements to an electrical current source, said electrical current source used to supply heat to the respective knee and foot cover members, said heat adjustment component also used to vary the amount of electrical current utilized so that the amount of heat supplied to the respective knee and foot cover members may be varied.

15. The knee and foot therapy apparatus as claimed in claim 11, said knee support member being hollow inside and capable of storing at least part of said electrical heating assembly.

16. The knee and foot therapy apparatus as claimed in claim 12, said foot support member being hollow inside and capable of storing at least part of said electrical heating assembly.

17. The knee and foot therapy apparatus as claimed in claim 10, said foot cover member being pivotally mounted to said foot support member such that said foot cover member may be positioned and secured at different angles.

18. The knee and foot therapy apparatus as claimed in claim 13, said support connection member being substantially adjustable such that said knee support member and said foot support member may be secured in close proximity to one another for storage and transport purposes.
